(19) United States
(12) Patent Application Publication
(10) Pub. No.: US 2025/0304210 A1
(43) Pub. Date: Oct. 2, 2025

(54) CONTROL DEVICE FOR HUMAN-POWERED VEHICLE, CONTROL SYSTEM FOR HUMAN-POWERED VEHICLE, AND HUMAN-POWERED VEHICLE HEAD PROTECTION DEVICE

(71) Applicant: Shimano Inc., Sakai (JP)

(72) Inventors: Mahiro HORIKAWA, Sakai (JP); Masataka YAMADA, Sakai (JP); Tomohiro KONDO, Sakai (JP); Takehiko NAKAJIMA, Sakai (JP)

(21) Appl. No.: 19/049,812

(22) Filed: Feb. 10, 2025

(30) Foreign Application Priority Data
Apr. 2, 2024 (JP) 2024-059573

(51) Int. Cl. B62M 6/45; A42B 3/04; A61B 5/00; A61B 5/11; A61B 5/117

(52) U.S. Cl. CPC B62M 6/45; A42B 3/0433; A61B 5/1126; A61B 5/6803; A61B 5/117; A61B 2562/0219

(57) ABSTRACT

A control device for a human-powered vehicle includes a controller. The controller is configured to control a component of the human-powered vehicle based on a worn state of a head protection device that is wearable by a rider of the human-powered vehicle.

### CROSS-REFERENCE TO RELATED APPLICATIONS

This application claims priority to Japanese Patent Application No. 2024-059573, filed on Apr. 2, 2024. The entire disclosure of Japanese Patent Application No. 2024-059573 is hereby incorporated herein by reference.

### BACKGROUND

### Technical Field

The present disclosure generally relates to a control device for a human-powered vehicle, a control system for a human-powered vehicle, and a human-powered vehicle head protection device.

### Background Information

Japanese Laid-Open Patent Publication No. 2010-047854 (Patent Document 1) discloses a helmet for a human-powered vehicle.

### SUMMARY

An objective of the present disclosure is to provide a control device for a human-powered vehicle, a control system for a human-powered vehicle, and a human-powered vehicle head protection device that improve convenience for a user.

A control device in accordance with a first aspect of the present disclosure is for a human-powered vehicle. The control device comprises a controller configured to control a component of the human-powered vehicle based on a worn state of a head protection device that is wearable by a rider of the human-powered vehicle.

With the control device according to the first aspect, the component of the human-powered vehicle is controlled based on the worn state of the head protection device. Thus, the controller performs appropriate control on the component in accordance with the worn state of the head protection device without the user having to operate the component based on the worn state of the head protection device. The control device improves convenience for the user.

In accordance with a second aspect of the present disclosure, the control device according to the first aspect further comprises a receiver configured to receive predetermined information related to the worn state from the head protection device. The controller is configured to control the component based on the predetermined information.

With the control device according to the second aspect, the worn state of the head protection device is appropriately determined based on the predetermined information.

In accordance with a third aspect of the present disclosure, the control device according to the first or second aspect is configured so that the component includes a restricting device configured to restrict traveling of the human-powered vehicle. The controller is configured to control the restricting device based on the worn state.

With the control device according to the third aspect, the traveling of the human-powered vehicle is restricted based on the worn state of the head protection device.

In accordance with a fourth aspect of the present disclosure, the control device according to any one of the first to third aspects is configured so that the component includes a motor configured to apply a propulsion force to the human-powered vehicle. The controller is configured to control the motor based on the worn state.

With the control device according to the fourth aspect, the motor is controlled based on the worn state of the head protection device.

In accordance with a fifth aspect of the present disclosure, the control device according to any one of the first to fourth aspects is configured so that the controller is configured to change an electric power consumption state of the component based on the worn state.

With the control device according to the fifth aspect, the electric power consumption state of the component is changed based on the worn state of the head protection device.

A control system in accordance with a sixth aspect of the present disclosure is for a human-powered vehicle. The control system comprises the control device according to any one of the first to fifth aspects, and a human-powered vehicle head protection device including a transmitter configured to transmit predetermined information related to the worn state to the control device.

With the control system according to the sixth aspect, the worn state of the head protection device is transmitted to the control device. Thus, the control device performs appropriate control on the component without the user having to input the worn state of the head protection device into the control device. The control system improves convenience for the user.

A human-powered vehicle head protection device in accordance with a seventh aspect of the present disclosure is wearable by a rider of a human-powered vehicle. The head protection device comprises a wear detector configured to detect a worn state of the head protection device that is worn by the rider, and a transmitter configured to transmit predetermined information related to the worn state, detected by the wear detector, to an external device.

With the head protection device according to the seventh aspect, the worn state of the head protection device is transmitted to an external device. Thus, the external device performs appropriate control on the worn state of the head protection device without the user having to input the worn state of the head protection device to the external device. The control system improves convenience for the user.

A human-powered vehicle head protection device in accordance with an eighth aspect of the present disclosure is wearable by a rider of a human-powered vehicle. The head protection device comprises a power supply, a wear detector configured to detect a worn state of the head protection device that is worn by the rider, a display configured to display an electric power level of the power supply, and a controller configured to control the display. The controller is configured to control the display to change a display state of the electric power level on the display based on the worn state detected by the wear detector.

With the head protection device according to the eighth aspect, the display state of the electric power level on the display is changed based on the worn state of the head protection device.

In accordance with a ninth aspect of the present disclosure, the head protection device according to the eighth aspect is configured so that the worn state includes a first worn state in which the head protection device is worn by the rider and a second worn state in which the head protection device is not worn by the rider. The controller is configured to control the display to change the display state in a case where the worn state is changed from one of the first worn state and the second worn state to the other one of the first worn state and the second worn state.

With the head protection device according to the ninth aspect, in a case where the worn state is changed from one of the first worn state and the second worn state to the other one of the first worn state and the second worn state, the display state is changed.

In accordance with a tenth aspect of the present disclosure, the head protection device according to the ninth aspect further comprises a motion detector configured to detect motion of the head protection device. The controller is configured to control the display to start displaying the electric power level in a case where motion of the head protection device is detected by the motion detector in the second worn state.

The head protection device according to the tenth aspect starts displaying the electric power level in the second worn state in a case where the motion detector detects motion of the head protection device. Thus, for example, in a case where the rider holds the head protection device, the rider can recognize the electric power level.

In accordance with an eleventh aspect of the present disclosure, the head protection device according to the tenth aspect is configured so that the motion detector includes at least one of an acceleration sensor and a gyro sensor.

With the head protection device according to the eleventh aspect, motion of the head protection device is appropriately detected by at least one of the acceleration sensor and the gyro sensor.

In accordance with a twelfth aspect of the present disclosure, the head protection device according to the eleventh aspect is configured so that in a case where the worn state changes from the second worn state to the first worn state, the controller is configured to control the display to finish displaying the electric power level.

The head protection device according to the twelfth aspect finishes displaying the electric power level in a case where the worn state is changed from the second worn state to the first worn state. Thus, electric power consumption is reduced.

In accordance with a thirteenth aspect of the present disclosure, the head protection device according to any one of the ninth to twelfth aspects is configured so that in a case where the worn state changes from the first worn state to the second worn state, the controller is configured to control the display to start displaying the electric power level.

The head protection device according to the thirteenth aspect starts displaying the electric power level in a case where the worn state is changed from the first worn state to the second worn state. Thus, in a case where the rider removes the head protection device, the rider acknowledges the electric power level.

A human-powered vehicle head protection device in accordance with a fourteenth aspect of the present disclosure is wearable by a rider of a human-powered vehicle. The head protection device comprises a wear detector configured to detect a worn state of the head protection device that is worn by the rider, an impact absorber, and a controller configured to control the impact absorber. The controller is configured to control the impact absorber based on the worn state detected by the wear detector.

The head protection device according to the fourteenth aspect controls the impact absorber based on the worn state.

In accordance with a fifteenth aspect of the present disclosure, in the head protection device according to the fourteenth aspect, the controller is configured to control the impact absorber in either one of a first actuation state in which the impact absorber is actuated and a second actuation state in which the impact absorber is actuated in a manner more limited than in the first actuation state. The controller is configured to control the impact absorber in the second actuation state in a case where the head protection device is not worn.

With the head protection device according to the fifteenth aspect, in a case where the head protection device is not worn, the impact absorber is configured to be controlled in the second actuation state. This avoids actuation of the impact absorber in a case where the head protection device is dropped.

In accordance with a sixteenth aspect of the present disclosure, the head protection device according to any one of the seventh to fifteenth aspects further comprises a wireless power receiver.

With the head protection device according to the sixteenth aspect, electric power is received by the wireless power receiver.

In accordance with a seventeenth aspect of the present disclosure, the head protection device according to the sixteenth aspect further comprises a position adjuster configured to adjust a position of the wireless power receiver relative to a wireless power transmitter that supplies electric power to the wireless power receiver.

With the head protection device according to the seventeenth aspect, the position adjuster allows the wireless power receiver to receive electric power in a preferred manner.

In accordance with an eighteenth aspect of the present disclosure, the head protection device according to any one of the seventh to seventeenth aspects further comprises a wired power receiver to which an electric power wire is connected.

With the head protection device according to the eighteenth aspect, electric power is received by the wired power receiver.

In accordance with a nineteenth aspect of the present disclosure, the head protection device according to any one of the seventh to eighteenth aspects is configured so that the wear detector is configured to detect a position of a head of the rider relative to the head protection device.

With the head protection device according to the nineteenth aspect, the worn state is appropriately detected by the position of the head of the rider relative to the head protection device.

In accordance with a twentieth aspect of the present disclosure, in the head protection device according to the nineteenth aspect, the wear detector is configured to detect the position based on information from a sensor that detects a reflected signal.

With the head protection device according to the twentieth aspect, the worn state is appropriately detected based on information from the sensor detecting a reflected signal.

In accordance with a twenty-first aspect of the present disclosure, the head protection device according to any one of the seventh to twentieth aspects further comprises a belt configured to attach the head protection device to the rider. The wear detector is configured to detect a coupled state of the belt.

With the head protection device according to the twenty-first aspect, the worn state is appropriately detected by the coupled state of the belt.

According to the present disclosure, the control device for a human-powered vehicle, the control system for a human-powered vehicle, and the human-powered vehicle head protection device improve convenience for a user.

### BRIEF DESCRIPTION OF THE DRAWINGS

Referring now to the attached drawings which form a part of this original disclosure, an illustrative embodiment is shown.

FIG. 1 is a side elevational view of a rider and a human-powered vehicle including a human-powered vehicle control system including a human-powered vehicle control device and a human-powered vehicle head protection device in accordance with a first embodiment.

FIG. 2 is a perspective view of the human-powered vehicle head protection device shown in FIG. 1.

FIG. 3 is a block diagram showing the electrical configuration of the human-powered vehicle control system shown in FIG. 1.

FIG. 4 is a flowchart of a control process for transmitting predetermined information executed by a second controller shown in FIG. 3.

FIG. 5 is a flowchart of a control process for changing a control state of a component executed by a first controller shown in FIG. 3.

FIG. 6 is a perspective view of a human-powered vehicle head protection device in accordance with a second embodiment.

FIG. 7 is a block diagram showing the electrical configuration of the human-powered vehicle head protection device of the second embodiment.

FIG. 8 is a first part of a flowchart of a control process for controlling a display executed by a second controller shown in FIG. 7.

FIG. 9 is a second part of the flowchart of the control process for controlling the display executed by the second controller shown in FIG. 7.

FIG. 10 is a perspective view of a human-powered vehicle head protection device in accordance with a third embodiment.

FIG. 11 is a block diagram showing the electrical configuration of the human-powered vehicle head protection device in the third embodiment.

FIG. 12 is a flowchart of a control process for controlling an impact absorber executed by the second controller shown in FIG. 11.

FIG. 13 is a flowchart of a control process for determining a worn state executed by the second controller shown in FIG. 11.

FIG. 14 is a block diagram showing the electrical configuration of a human-powered vehicle head protection device in accordance with a fourth embodiment.

FIG. 15 is a perspective view of a human-powered vehicle head protection device coupled to a holder in the fourth embodiment.

FIG. 16 is a perspective view of a human-powered vehicle head protection device in accordance with a first modification.

FIG. 17 is a block diagram showing the electrical configuration of a human-powered vehicle head protection device in accordance with a second modification.

### DETAILED DESCRIPTION OF THE EMBODIMENTS

Selected embodiments will now be explained with reference to the drawings. It will be apparent to those skilled in the bicycle field from this disclosure that the following descriptions of the embodiments are provided for illustration only and not for the purpose of limiting the invention as defined by the appended claims and their equivalents.

### First Embodiment

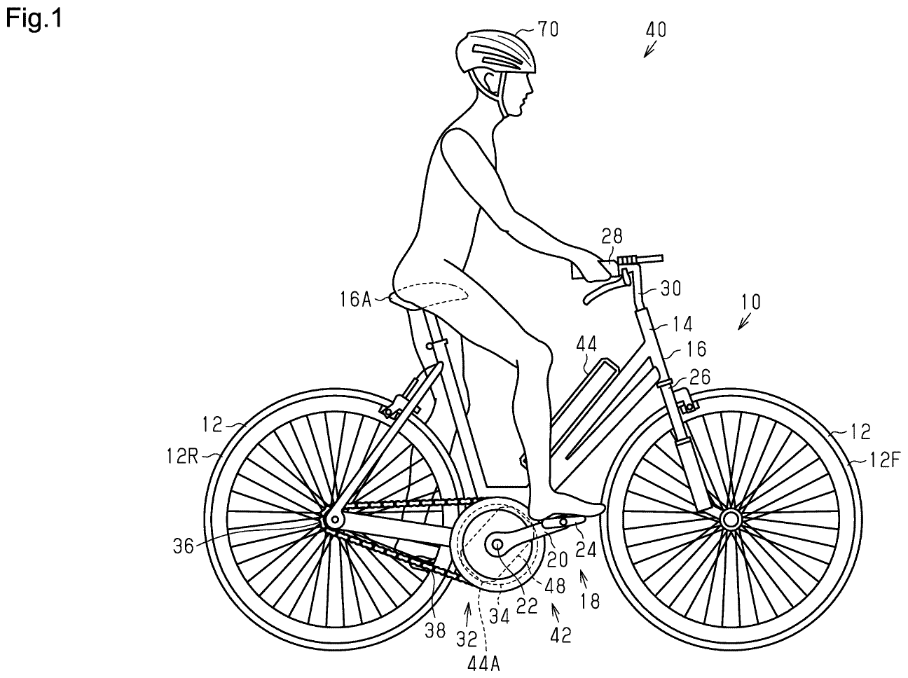

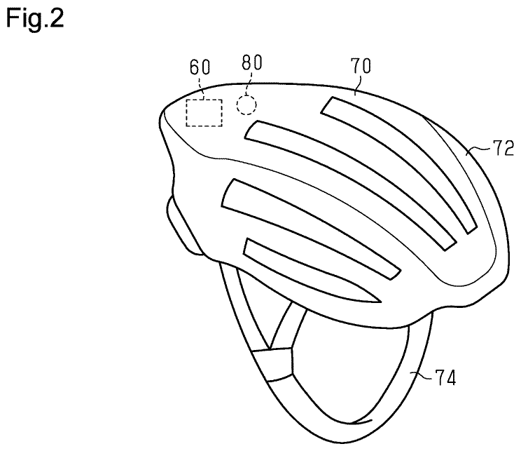

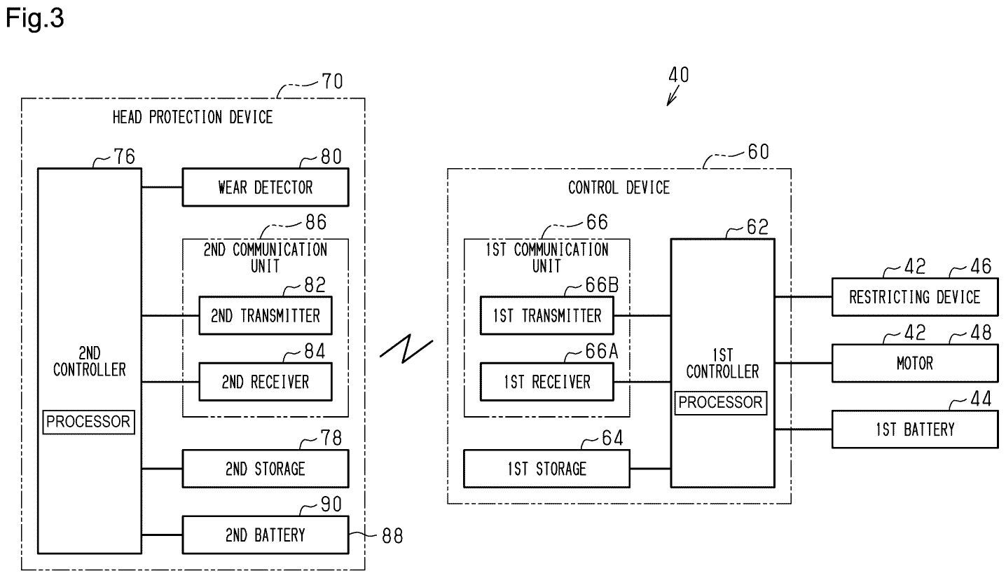

Referring initially to FIG. 1\, a human-powered vehicle 10 is illustrated in which a human-powered vehicle control system 40 is applied in accordance with a first embodiment. The human-powered vehicle control system 40 basically comprises a human-powered vehicle control device 60 and a human-powered vehicle head protection device 70. With reference to FIGS. 2 to 5, the human-powered vehicle control system 40, the human-powered vehicle control device 60 and the human-powered vehicle head protection device 70 will be described.

The human-powered vehicle is a vehicle including at least one wheel and driven by at least a human driving force. The human-powered vehicle includes, for example, various types of bicycles such as a mountain bike, a road bike, a city bike, a cargo bike, a hand bike, and a recumbent bike. The number of wheels on the human-powered vehicle is not limited. The human-powered vehicle includes, for example, a unicycle and a vehicle including two or more wheels. The human-powered vehicle is not limited to a vehicle configured to be driven only by a human driving force. The human-powered vehicle includes an E-bike that uses a driving force of an electric motor in addition to a human driving force for propulsion. The E-bike includes an electric assist bicycle that assists in propulsion with an electric motor. In the embodiments described below, the human-powered vehicle refers to an electric assist bicycle.

Here, the human-powered vehicle 10 includes at least one wheel 12 and a body 14. The at least one wheel 12 includes, for example, a front wheel 12F and a rear wheel 12R. The body 14 includes a frame 16. In an example, a saddle 16A is coupled to the frame 16.

In an example, the human-powered vehicle 10 further includes a crank 18 into which the human driving force is input. In an example, the crank 18 includes a crank arm 20 and a crank axle 22. The crank axle 22 is, for example, rotatable relative to the frame 16. In an example, a pedal 24 is coupled to the crank arm 20. The crank arm 20 is provided, for example, on each axial end of the crank axle 22.

A front fork 26 is joined to the frame 16. The front wheel 12F is attached to the front fork 26. A handlebar 28 is coupled to the front fork 26 by a stem 30. The rear wheel 12R is supported by the frame 16. In the present embodiment, the crank 18 is connected to the rear wheel 12R by a drive mechanism 32. The rear wheel 12R is driven in accordance with rotation of the crank axle 22. Any one of the front wheel 12F and the rear wheel 12R can be coupled to the crank 18 by the drive mechanism 32.

The drive mechanism 32 includes at least one first rotational body 34 coupled to the crank axle 22. In an example, the at least one first rotational body 34 includes a front sprocket. The at least one first rotational body 34 can include a pulley or a bevel gear. The crank axle 22 can be coupled to the front sprocket by a one-way clutch.

The drive mechanism 32 further includes at least one second rotational body 36 and a transferring member 38. The transferring member 38 is configured to transmit a rotational force of the at least one first rotational body 34 to the at least one second rotational body 36. In an example, the transferring member 38 includes a chain. The transferring member 38 can include a belt or a shaft. In an example, the at least one second rotational body 36 includes a rear sprocket. The at least one second rotational body 36 can include a pulley or a bevel gear. The chain is wound around, for example, the front sprocket and the rear sprocket. The at least one second rotational body 36 is coupled, for example, to the rear wheel 12R. In an example, the rear wheel 12R is configured to rotate in accordance with the rotation of the at least one second rotational body 36.

As mentioned above, the human-powered vehicle control system 40 includes the human-powered vehicle control device 60 and the human-powered vehicle head protection device 70. The human-powered vehicle control system 40 further includes, for example, a human-powered vehicle component 42.

The human-powered vehicle control device 60 includes a controller 62. In the present embodiment, the controller 62 of the control device 60 can be referred to as a first controller 62 or a first electronic controller 62. The first controller 62 includes, for example, at least one processor that executes a predetermined control program. The processor of the first controller 62 includes, for example, a central processing unit (CPU) or a micro-processing unit (MPU). The first controller 62 is formed of one or more semiconductor chips that are mounted on a printed circuit board. The terms “controller” and “electronic controller” as used herein refer to hardware that executes a software program, and does not include a human being. The first controller 62 is, for example, configured to control the component 42.

In an example, the processor of the first controller 62 can include parts arranged at separate locations. In a case where the parts of the processor are arranged at separate locations, the parts of the processor can be connected so as to communicate with each other via a wireless communication device. The first controller 62 can include one or more microcomputers.

The control device 60 further includes, for example, a first storage 64. In an example, the first storage 64 is connected to the first controller 62 through wired or wireless communication. In an example, the first storage 64 stores control programs and information used for control processing. In an example, the first storage 64 includes a non-volatile memory and a volatile memory. The non-volatile memory includes, for example, at least one of a read-only memory (ROM), an erasable programmable read only memory (EPROM), an electrically erasable programmable read-only memory (EEPROM), and a flash memory. The volatile memory includes, for example, a random access memory (RAM).

The control system 40 further includes, for example, a battery 44 configured to supply electric power to the control device 60. In the present embodiment, the battery 44 configured to supply electric power to the control device 60 can be referred to as a first battery 44. The first battery 44 includes one or more battery elements. The battery element includes a rechargeable battery. In an example, the first battery 44 is configured to supply electric power to the control device 60 and the component 42. In an example, the first battery 44 is connected to the control device 60 through wired or wireless communication. In an example, the first battery 44 is configured to communicate with the control device 60 through, power line communication (PLC), controller area network (CAN), or universal asynchronous receiver/transmitter (UART).

The component 42 includes, for example, a restricting device 46 configured to restrict traveling of the human-powered vehicle 10. The restricting device 46 is, for example, configured to restrict rotation of the wheel 12 and at least one rotational body of the drive mechanism 32. The restricting device 46 can include a braking device that brakes the wheel 12. The braking device 46 can be, for example, a rim brake (e.g., a caliper brake, a V-brake or a cantilever brake), a disc brake, a drum brake or a roller brake. The restricting device 46 is, for example, configured to switch the state of the restricting device 46 between a restricted state in which traveling of the human-powered vehicle 10 is restricted and a release state in which the traveling of the human-powered vehicle 10 is unrestricted. The first controller 62 is, for example, configured to control the restricting device 46 so that the state of the restricting device 46 is switched between the restricted state and the release state. In an example, in a case where the restricting device 46 includes a key hole and a release operation is performed on the key hole with a key, the first controller 62 controls the restricting device 46 so that the state of the restricting device 46 is switched from the restricted state to the release state. In an example, in a case where an operating device receives release information, the first controller 62 controls the restricting device 46 to switch the state of the restricting device 46 from the restricted state to the release state. The release information includes, for example, at least one of a password and biometric authentication of a user.

In an example, the component 42 includes a motor 48 that applies a propulsion force to the human-powered vehicle 10. In a case where the component 42 includes the motor 48, the control device 60 can further include a drive circuit of the motor 48. In an example, the first controller 62 and the drive circuit are provided on the motor 48. The controller 62 and the drive circuit can be provided on the same circuit board. In an example, the drive circuit is connected to the first controller 62 through wired or wireless communication. In an example, the drive circuit drives the motor 48 in response to a control signal from the first controller 62.

In an example, the drive circuit is electrically connected to the motor 48. In an example, the drive circuit controls the supply of electric power from the first battery 44 to the motor 48. In an example, the drive circuit includes an inverter circuit. In an example, the inverter circuit includes transistors. In an example, the inverter circuit has a configuration in which inverter units are connected in parallel and each inverter unit is formed by two transistors connected in series. In an example, the inverter circuit can include a current sensor that detects the current flowing through the inverter circuit. In an example, the current sensor is connected to the first controller 62 through wired or wireless communication.

The first controller 62 is configured to control the motor 48. In an example, the first controller 62 is configured to control the motor 48 in accordance with a state of the human-powered vehicle 10. In an example, the first controller 62 is configured to control the motor 48 to change output of the motor 48 in accordance with the human driving force that is applied to the human-powered vehicle 10. In an example, the first controller 62 is configured to control the motor 48 to change a propulsion force in accordance with the human driving force. In an example, the first controller 62 is configured to control the motor 48 in accordance with the human driving force that is detected by a human driving force detector.

In an example, the human-powered vehicle 10 further includes the human driving force detector for detecting a traveling condition or state of the human-powered vehicle 10. The term “detector” as used herein refers to a hardware device or instrument designed to detect the presence or absence of a particular event, object, substance, or a change in its environment, and to emit a signal in response. The term “detector” as used herein does not include a human being. In an example, the human driving force detector is connected to the first controller 62 through wired or wireless communication. In an example, the human driving force detector is configured to output a signal corresponding to a torque that is applied to the crank axle 22 by the human driving force. The signal corresponding to torque applied to the crank axle 22 by human driving force includes information related to the human driving force applied to the human-powered vehicle 10.

In an example, the human driving force detector is provided on a member included in the transmission path of the human driving force or a member near the member included in the transmission path of the human driving force. The human driving force detector includes a strain sensor, a magnetostrictive sensor, a pressure sensor, or the like. The strain sensor includes a strain gauge. The human driving force detector can have any configuration that obtains information related to a human driving force.

In an example, the human driving force detector can be provided on at least one of the crank arm 20 and the pedal 24. In a case where the human driving force detector is provided on the pedal 24, the human driving force detector can include a sensor that detects pressure applied to the pedal 24. The human driving force detector can be provided on the chain. In a case where the human driving force detector is provided on the chain, the human driving force detector can include a sensor that detects tension on the chain.

For example, the human-powered vehicle 10 further includes a vehicle speed detector. In an example, the vehicle speed detector is connected to the first controller 62 through wired or wireless communication. The vehicle speed detector is configured to detect, for example, information related to the speed of the human-powered vehicle 10. The vehicle speed detector is configured to detect, for example, information related to the rotational speed of the wheel 12. The vehicle speed detector is configured to detect, for example, a magnet provided on at least one of the front wheel 12F and the rear wheel 12R.

The vehicle speed detector is, for example, configured to output a predetermined number of detection signals during a period in which the wheel 12 completes one rotation. The predetermined number is, for example, one. The vehicle speed detector outputs, for example, a signal corresponding to the rotational speed of the wheel 12. The first controller 62 calculates the speed of the human-powered vehicle 10 based on the signal corresponding to the rotational speed of the wheel 12 and information related to the circumferential length of the wheel 12. The information related to the circumferential length of the wheel 12 is stored in, for example, the first storage 64.

In an example, the human-powered vehicle 10 further includes a crank rotation state detector. The crank rotation state detector is provided, for example, on a drive unit 44A. In an example, the crank rotation state detector is connected to the first controller 62 through wired or wireless communication.

The crank rotation state detector is, for example, configured to detect a rotational amount of the crank axle 22 and a rotational amount of the first rotational body 34. The first rotational body 34 includes, for example, a front sprocket or a front pulley. The crank rotation state detector is, for example, configured to detect at least one of information corresponding to the rotational speed of the crank axle 22 and information corresponding to the rotational speed of the first rotational body 34. The information related to the rotational speed of the crank axle 22 includes, for example, angular acceleration of the crank axle 22. The information corresponding to the rotational speed of the first rotational body 34 includes, for example, angular acceleration of the first rotational body 34.

In an example, the crank rotation state detector is configured to output at least one of a signal corresponding to the rotational speed of the crank axle 22 and a signal corresponding to the rotational speed of the first rotational body 34. In an example, the crank rotation state detector is configured to output at least one of a detection signal corresponding to a rotational angle of the crank axle 22 and a detection signal corresponding to a rotational angle of the first rotational body 34 during one rotation of the crank axle 22 and the first rotational body 34.

In an example, the crank rotation state detector includes a magnetic sensor that outputs a signal corresponding to the strength of a magnetic field. In an example, the crank rotation state detector includes an annular magnet having a plurality of magnetic poles arranged in the circumferential direction. In an example, the annular magnet is provided on the crank axle 22. The annular magnet includes, for example, a single S-pole and a single N-pole. The S-pole and the N-pole each continuously extend 180° about the rotational center axis of the crank axle 22 in the circumferential direction. The crank rotation state detector can include, for example, an optical sensor, an acceleration sensor, a gyro sensor, or a torque sensor instead of the magnetic sensor.

The crank rotation state detector can be configured to detect a rotational amount of the second rotational body 36. The second rotational body 36 includes, for example, a rear sprocket or a rear pulley. The crank rotation state detector can be configured to detect information corresponding to the rotational speed of the second rotational body 36. The information corresponding to the rotational speed of the second rotational body 36 includes, for example, angular acceleration of the second rotational body 36. The crank rotation state detector can be configured to output a signal corresponding to the rotational speed of the second rotational body 36.

The crank rotation state detector can be configured to include a vehicle speed sensor. In a case where the crank rotation state detector includes a vehicle speed sensor, the first controller 62 can be configured to calculate the rotational speed of the crank axle 22 from the vehicle speed detected by the vehicle speed sensor and the transmission ratio.

In an example, the first controller 62 is configured to control the motor 48 in accordance with at least one of the rotational speed of the crank axle 22 and the rotational speed of the first rotational body 34 that are detected by the crank rotation state detector. For example, the first controller 62 is configured to control the motor 48 in accordance with the speed of the human-powered vehicle 10 detected by a vehicle speed detector.

In an example, in a case where the speed of the human-powered vehicle 10 is less than a predetermined vehicle speed, the first controller 62 is configured to drive the motor 48 so as to apply a propulsion force to the human-powered vehicle 10 in accordance with at least one of the human driving force and the rotational speed of the crank axle 22. The predetermined vehicle speed is, for example, determined by regulations in each country. The predetermined vehicle speed is, for example, 24 km/h, 25 km/h, 30 km/h, 32 km/h, or 45 km/h.

The first controller 62 is, for example, configured to control the motor 48 so that an assist force produced by the motor 48 equals a predetermined assist force. The assist force includes, for example, at least one of the ratio of output of the motor 48 to the human driving force input to the human-powered vehicle 10 and the upper limit value of output of the motor 48.

The human driving force is, for example, expressed as at least one of torque and power. In a case where the human driving force is expressed as torque, the human driving force is referred to as, for example, human torque. In a case where the human driving force is expressed as power, for example, the human driving force is referred to as human force-based power. The human force-based power is, for example, the product of the torque applied to the crank axle 22 and the rotational speed of the crank axle 22.

The assist force is, for example, expressed as at least one of torque and power. In a case where the assist force is expressed as torque, the assist force is referred to as, for example, assist torque. In a case where the assist force is expressed as power, the assist force is referred to as, for example, assist power. The ratio of the assist force to the human driving force can be a ratio of the assist torque to the human torque or a ratio of the assist power to the human force-based power.

The control device 60 further includes a receiver 66A configured to receive predetermined information related to the worn state of the head protection device 70 from the head protection device 70. In the present embodiment, the receiver 66A of the control device 60 can be referred to as a first receiver 66A. The control device 60 can further include a first transmitter 66B configured to transmit information to the head protection device 70. The first receiver 66A and the first transmitter 66B form, for example, a first communication unit 66 (i.e., a first communicator). The first communication unit 66 is, for example, configured to perform wireless communication. In this case, the first communication unit 66 can be referred to as a first wireless communicator.

The head protection device 70 is, for example, a helmet wearable by a rider of the human-powered vehicle 10. In a case where the human-powered vehicle 10 includes a seat for a person other than the driver such as a child seat, the rider includes at least one of a driver and a passenger. The head protection device 70 can include anything that protects the head of the rider in addition to a helmet. In an example, the head protection device 70 can include an air bag that inflates to cover the head of the rider in a case where at least one of the rider and the human-powered vehicle 10 receives an impact. In a case where the head protection device 70 includes an air bag, the head protection device 70 can be configured to be worn on the neck or the back of the rider.

The human-powered vehicle head protection device 70 is wearable by the rider of the human-powered vehicle 10. The head protection device 70 includes, for example, a main body 72. The main body 72 is configured to be worn on the body of the rider. The head protection device 70 further includes a strap or a belt 74 configured to attach the head protection device 70 to the rider. In a case where the head protection device 70 includes a helmet, the belt 74 is, for example, configured to be put around the chin of the rider.

The head protection device 70 includes, for example, a controller 76. In the present embodiment, the controller 62 of the head protection device 70 can be referred to as a second controller 76 or a second electronic controller 76. The second controller 76 includes, for example, at least one processor that executes a predetermined control program. The processor of the second controller 76 includes, for example, a CPU or MPU.

In an example, the processor of the second controller 76 can include parts arranged at separate locations. In a case where the parts of the processor are arranged at separate locations, the parts of the processors can be connected so as to communicate with each other via a wireless communication device. The second controller 76 can include one or more microcomputers.

The head protection device 70 further includes, for example, second storage 78. In an example, the second storage 78 is connected to the second controller 76 through wired or wireless communication. In an example, the second storage 78 stores control programs and information used for control processing. In an example, the second storage 78 includes a non-volatile memory and a volatile memory. The non-volatile memory includes, for example, at least one of a ROM, an EPROM, an EEPROM, and a flash memory. The volatile memory includes, for example, a RAM.

The head protection device 70 includes, for example, a wear detector 80 configured to detect a worn state of the head protection device 70 and a transmitter 82 configured to transmit predetermined information related to the worn state, detected by the wear detector 80, to an external device. The external device includes the control device 60. Instead of or in addition to the control device 60, the external device can include at least one of a personal computer, a smartphone, a tablet computer, and a server.

The wear detector 80 outputs, for example, a signal corresponding to the worn state of the head protection device 70 on the rider. The wear detector 80 is, for example, provided on a portion of the main body 72 facing the body of the rider. The wear detector 80 is, for example, configured to detect a position of the head of the rider relative to the head protection device 70. The wear detector 80 can be, for example, configured to detect the position of the head of the rider relative to the head protection device 70 based on information from a sensor configured to detect a reflected signal. The wear detector 80 includes, for example, a signal illuminator and a signal detector. The signal illuminator is, for example, configured to emit light. The signal detector is, for example, configured to receive the reflected light. The wear detector 80 is, for example, configured to detect at least one of the body of the rider and the distance to the body of the rider. The wear detector 80 includes, for example, at least one of an optical sensor, an ultrasonic sensor, and a distance sensor. The wear detector 80 can be configured to output a signal corresponding to contact of the inner surface of the main body 72 with the head of the rider. In a case where the wear detector 80 is configured to output a signal corresponding to contact of the inner surface of the main body 72 with the head of the rider, the wear detector 80 includes, for example, at least one of a pressure sensor and a switch. The wear detector 80 can be configured to detect a coupled state of the belt 74. The wear detector 80 is, for example, configured to detect an attachment state of the belt 74. For example, in a case where the belt 74 includes a free end, the attachment state of the belt 74 includes an attachment state of the free end of the belt 74 to a belt coupling portion. The belt coupling portion is, for example, provided on the main body 72 or a free end of an additional belt that is provided on the main body 72.

The head protection device 70 includes, for example, the transmitter 82 configured to transmit predetermined information related to the worn state to the control device 60. In the present embodiment, the transmitter 82 of the head protection device 70 can be referred to as a second transmitter 82. The head protection device 70 can further include a second receiver 84 configured to receive information. The second transmitter 82 and the second receiver 84 form, for example, a second communication unit 86 (i.e., a second communicator). The second communication unit 86 is, for example, configured to perform wireless communication. In this case, the second communication unit 86 can be referred to as a second wireless communicator.

The head protection device 70 further includes, for example, a power supply 88 configured to supply electric power to the second controller 76. The power supply 88 includes, for example, a second battery 90. The second battery 90 includes one or more battery elements. The battery element includes a rechargeable battery. In an example, the second battery 90 is configured to supply electric power to the second controller 76, the wear detector 80, and the second communication unit 86. The second battery 90 can be connected to the second controller 76 to perform wired or wireless communication with the second controller 76. In an example, the second battery 90 communicates with the second controller 76 through power line communication, CAN, or UART.

The worn state includes, for example, a first worn state in which the head protection device 70 is worn by the rider and a second worn state in which the head protection device 70 is not worn by the rider. The predetermined information includes, for example, information indicating whether the worn state is the first worn state or the second worn state. The second controller 76 is configured to control the second transmitter 82 to transmit the predetermined information, for example, in a case where the worn state is changed. The predetermined information includes at least one of information indicating the worn state is the first worn state and information indicating that the worn state is the second worn state. The predetermined information can include at least one of attachment information indicating that the worn state is changed from the second worn state to the first worn state and removal information indicating that the worn state is changed from the first worn state to the second worn state. In an example, in a case where the worn state is changed from the first worn state to the second worn state, the second controller 76 is configured to control the second transmitter 82 to transmit the removal information. In an example, in a case where the second receiver 84 receives a transmission request for predetermined information from the control device 60, the second controller 76 is configured to control the second transmitter 82 to transmit the predetermined information.

In a case where the wear detector 80 is configured to detect a position of the head of the rider relative to the head protection device 70, the second controller 76 determines that the worn state is the first state, for example, if the head of the rider is located within a predetermined distance from the head protection device 70. In a case where the wear detector 80 is configured to detect a position of the head of the rider relative to the head protection device 70, the second controller 76 determines that the worn state is the second state, for example, if the head of the rider is located outside the predetermined distance from the head protection device 70. In a case where the wear detector 80 is configured to detect a signal corresponding to contact of the inner surface of the main body 72 with the head of the rider, the second controller 76 determines that the worn state is the first state, for example, if an output signal of the wear detector 80 corresponds to a state in which the inner surface of the main body 72 is in contact with the head of the rider. In a case where the wear detector 80 is configured to detect a signal corresponding to contact of the inner surface of the main body 72 with the head of the rider, the second controller 76 determines that the worn state is the second state, for example, if an output signal of the wear detector 80 corresponds to a state in which the inner surface of the main body 72 is not in contact with the head of the rider. In a case where the wear detector 80 is configured to detect the coupled state of the belt 74, the second controller 76 determines that the worn state is the first state, for example, if the belt 74 is coupled to a belt attachment. In a case where the wear detector 80 is configured to detect the coupled state of the belt 74, the second controller 76 determines that the worn state is the second state, for example, if the belt 74 is not coupled to the belt attachment.

The controller 62 of the control device 60 is, for example, configured to control the human-powered vehicle component 42 based on the worn state of the head protection device 70 wearable by the rider of the human-powered vehicle 10. The controller 62 is, for example, configured to control the component 42 so that the control state of the component 42 is changed between a first control state and a second control state based on the worn state. The controller 62 of the control device 60 is, for example, configured to control the component 42 based on the predetermined information. The controller 62 is, for example, configured to control the component 42 so that the control state of the component 42 is changed between the first control state and the second control state based on the predetermined information. In an example, in a case where the worn state is the first worn state, the controller 62 sets the control state of the component 42 to the first control state. In an example, in a case where the worn state is the second worn state, the controller 62 sets the control state of the component 42 to the second control state.

In a case where the component 42 includes the restricting device 46, for example, the controller 62 is configured to control the restricting device 46 based on the worn state. In a case where the component 42 includes the restricting device 46, the first control state includes, for example, a release state. In a case where the component 42 includes the restricting device 46, the second control state includes, for example, a restricted state. In an example, in a case where the worn state is the first worn state, the first controller 62 is configured to control the restricting device 46 to switch the state of the restricting device 46 from the restricted state to the release state. In an example, in a case where the worn state is the second worn state, the first controller 62 is configured to control the restricting device 46 so that the restricting device 46 is maintained in the restricted state. In an example, in a case where the worn state is the second worn state, the first controller 62 is configured to control the restricting device 46 so that the restricting device 46 maintains the restricted state even if the release operation with the key is performed or the release information is received.

In a case where the component 42 includes the restricting device 46, the controller 62 is, for example, configured to control the motor 48 based on the worn state. In a case where the component 42 includes the motor 48, the first controller 62 is configured to control the motor 48, for example, so that the assist force of the motor 48 is less in the second control state than in the first control state. In an example, in a case where the worn state is the second worn state, the first controller 62 is configured to control the motor 48 so that the motor 48 does not apply a propulsion force to the human-powered vehicle 10. In an example, the first controller 62 is configured to control the motor 48 so that in a case where the worn state is the second worn state, the assist force of the motor 48 is less than in a case where the worn state is the first worn state.

The controller 62 can be configured to change an electric power consumption state of the component 42 based on the worn state. The electric power consumption state includes, for example, a first electric power consumption state and a second electric power consumption state in which less electric power is consumed than the first electric power consumption state. The first control state includes, for example, the first electric power consumption state. The second control state includes, for example, the second electric power consumption state. The second electric power consumption state corresponds to, for example, a sleep mode. For example, in a case where the electric power consumption state is the second electric power consumption state, at least some of functions of the component 42 are restricted more than in a case where the electric power consumption state is the first electric power consumption state. In an example, in a case where the worn state is the first worn state, the first controller 62 sets the electric power consumption state to the first electric power consumption state. In an example, in a case where the worn state is the second worn state, the first controller 62 sets the electric power consumption state to the second electric power consumption state. In an example, in the second electric power consumption state, in a case where the worn state is changed from the second worn state to the first worn state, the first controller 62 switches the electric power consumption state from the second electric power consumption state to the first electric power consumption state. In an example, in the first electric power consumption state, in a case where the worn state is changed from the first worn state to the second worn state, the first controller 62 switches the electric power consumption state from the first electric power consumption state to the second electric power consumption state.

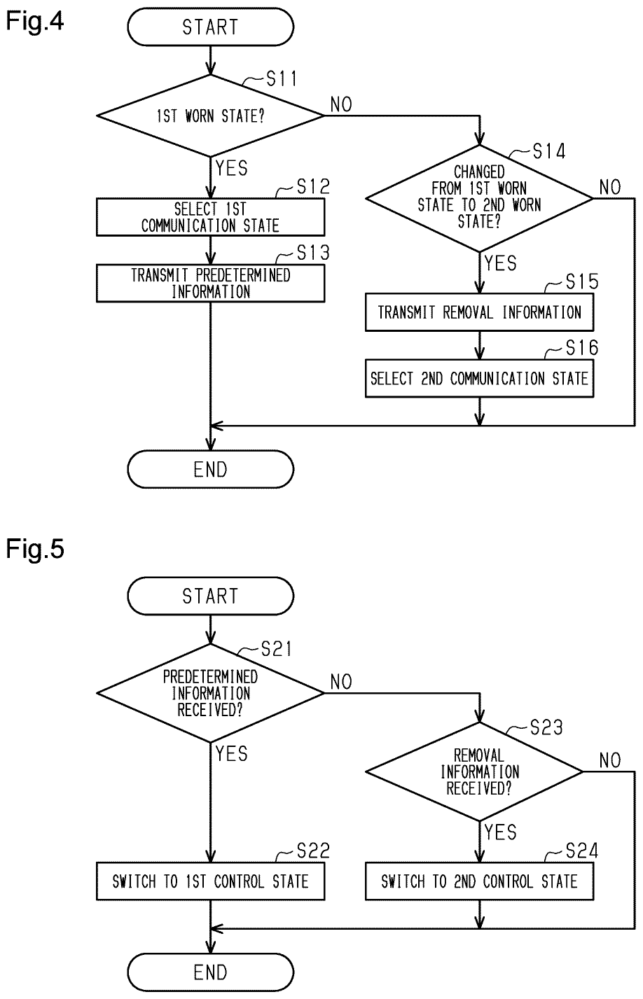

With reference to FIG. 4, a control process for transmitting the predetermined information with the second controller 76 will be described. For example, in a case where electric power is supplied to the second controller 76, the second controller 76 starts the control process of the flowchart shown in FIG. 4 from step S11. In a case where the flowchart shown in FIG. 4 ends, for example, the second controller 76 repeats the control process from step S11 after a predetermined interval until the supply of electric power stops.

In step S11, the second controller 76 determines whether the worn state is the first worn state. In a case where the worn state is the first worn state, the second controller 76 proceeds to step S12. In step S12, the second controller 76 selects a first communication state from communication states of the second communication unit 86 with the first communication unit 66 and proceeds to step S13. The first communication state is, for example, a state in which the second communication unit 86 is allowed to communicate with the first communication unit 66. For example, in step S12, in a case where the communication state of the second communication unit 86 with the first communication unit 66 is a second communication state, the controller 76 switches the communication state from the second communication state to the first communication state. In step S13, the second controller 76 transmits the predetermined information and ends the control process. The predetermined information that is transmitted in step S13 includes information indicating that the worn state is the first worn state.

In step S11, in a case where the worn state is not the first worn state, the second controller 76 proceeds to step S14. In step S14, the second controller 76 determines whether the worn state is changed from the first worn state to the second worn state. In a case where the worn state is changed from the first worn state to the second worn state, the second controller 76 proceeds to step S15. In step S15, the second controller 76 transmits the removal information and proceeds to step S16. In step S16, the controller 76 selects the second communication state from the communication states of the second communication unit 86 with the first communication unit 66 and then ends the control process. The second communication state is, for example, a state in which the second communication unit 86 does not communicate with the first communication unit 66. For example, in step S16, the controller 76 switches the communication state of the second communication unit 86 with the first communication unit 66 from the first communication state to the second communication state.

In step S14, in a case where the worn state is not changed from the first worn state to the second worn state, the second controller 76 ends the control process. The case in which the second controller 76 determines that the worn state is not changed from the first worn state to the second worn state includes, for example, a state where the worn state is maintained as the second worn state.

In the control process shown in FIG. 4, in a case where the worn state is the first worn state, the second controller 76 can be configured to transmit predetermined information indicating that the worn state is the first worn state to the control device 60 in each predetermined cycle. In this case, the second controller 76 can be configured to control the second transmitter 82 so that the predetermined information is transmitted to the control device 60 only in a case where the worn state is changed from the second worn state to the first worn state. Alternatively, the second controller 76 can be configured to control the second transmitter 82 so that the predetermined information is transmitted to the control device 60 only once in a case where the worn state is changed from the second worn state to the first worn state. In an example, instead of executing step S11, the second controller 76 is configured to determine that the worn state is changed from the second worn state to the first worn state. In an example, in a case where the worn state is changed from the second worn state to the first worn state, the second controller 76 proceeds to step S12. In an example, in a case where the worn state is changed from the second worn state to the first worn state, the second controller 76 can be configured to transmit the attachment information and then proceed to step S12. In an example, in a case where the worn state is not changed from the second worn state to the first worn state, the second controller 76 proceeds to step S14.

With reference to FIG. 5, a control process for changing the control state of the component 42 with the first controller 62 will be described. For example, in a case where electric power is supplied to the first controller 62, the first controller 62 starts the control process and proceeds to step S21 of the flowchart shown in FIG. 5. In a case where the flowchart shown in FIG. 5 ends, the first controller 62 repeats the control process from step S21 after a predetermined interval, for example, until the supply of electric power stops.

In step S21, the first controller 62 determines whether the predetermined information is received. In an example, in a case of receiving information indicating that the worn state is the first worn state or predetermined information including the attachment information, the first controller 62 determines that the predetermined information is received. In a case where the predetermined information is received, the first controller 62 proceeds to step S22. In step S22, the first controller 62 switches the control state to the first control state and ends the control process.

In step S21, in a case where the predetermined information is not received, the first controller 62 proceeds to step S23. In step S23, the first controller 62 determines whether the removal information is received. In a case where the removal information is received, the first controller 62 proceeds to step S24. In step S24, the first controller 62 switches the control state to the second control state and ends the control process. In step S23, in a case where the removal information is not received, the first controller 62 ends the control process.

In an example, instead of executing step S23, the first controller 62 can be configured to determine whether predetermined information indicating that the worn state is the second worn state is received. In a case where the predetermined information indicating that the worn state is the second worn state is received, the first controller 62 proceeds to step S24. In a case where the predetermined information indicating that the worn state is the second worn state is not received, the first controller 62 ends the control process.

### Second Embodiment

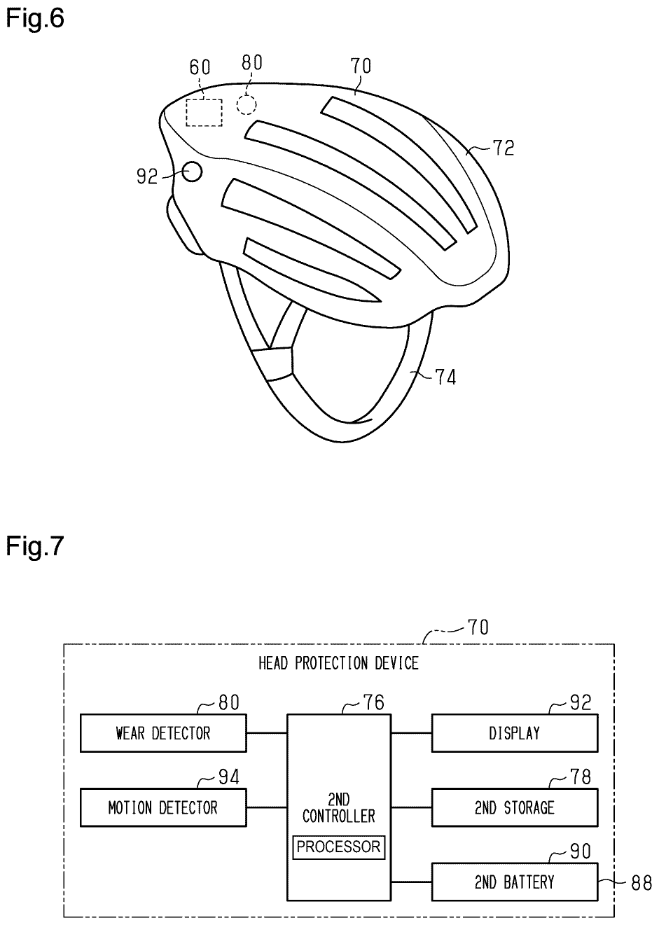

With reference to FIGS. 6 to 9, a second embodiment of the human-powered vehicle head protection device 70 will be described. In the head protection device 70 of the second embodiment, same reference characters are given to those elements that are the same as the corresponding elements in the first embodiment. Such elements will not be described in detail.

The head protection device 70 of the present embodiment includes the power supply 88, the wear detector 80, a display 92, and the controller 76. The display 92 is configured to display the electric power level of the power supply 88. The controller 76 is configured to control the display 92. The controller 76 is configured to control the display 92 to change the display state of the electric power level on the display 92 based on the worn state detected by the wear detector 80. In the present embodiment, the second communication unit 86 can be omitted from the head protection device 70.

The display 92 is, for example, provided on an outer surface of the head protection device 70. The display 92 is, for example, provided on the main body 72 at a position where the rider can see the display 92 in a state where the rider is not wearing the head protection device 70. The display 92 includes, for example, a light emitting portion. The light emitting portion includes, for example, a light emitting diode (LED). The display 92 can include a liquid crystal display. In a case where the display 92 includes a light emitting portion, the electric power level is, for example, indicated by at least one of an illuminated state, an intermittent illumination state, an unilluminated state, and an illumination color.

In an example, the head protection device 70 further includes a motion detector 94 configured to detect motion of the head protection device 70. The motion detector 94 is, for example, configured to detect a case where the head protection device 70 is in a rest state. The rest state is, for example, a state in which the head protection device 70 is located at a storage position. The motion detector 94 includes, for example, at least one of an acceleration sensor and a gyro sensor.

The controller 76 is configured to control the display 92 to change the display state, for example, in a case where the worn state is changed from one of the first worn state and the second worn state to the other one of the first worn state and the second worn state. The second controller 76 is configured to control the display 92 to display the electric power level, for example, in a case where the worn state is changed from one of the first worn state and the second worn state to the other one of the first worn state and the second worn state. The second controller 76 is configured to control the display 92 to display the electric power level, for example, in a case where the worn state is changed from the first worn state to the second worn state. In a case where the worn state changes from the first worn state to the second worn state, for example, the second controller 76 is configured to control the display 92 so that the state is changed from the unilluminated state to one of the illuminated state and the intermittent illumination state. In an example, in a case where the rider prepares the head protection device 70 to wear the head protection device 70 and a case where the rider removes the head protection device 70 from the body, the second controller 76 is configured to control the display 92 to display the electric power level.

The controller 76 is, for example, configured to control the display 92 to start displaying the electric power level in a case where motion of the head protection device 70 is detected by the motion detector 94 in the second worn state. The second controller 76 is, for example, configured to control the display 92 to start displaying the electric power level in a case where the head protection device 70 is changed from the rest state to a state differing from the rest state. The controller 76 is, for example, configured to control the display 92 to finish displaying the electric power level in a case where the worn state is changed from the second worn state to the first worn state. The second controller 76 is, for example, configured to control the display 92 so as not to start displaying the electric power level even in a case where the motion detector 94 detects motion of the head protection device 70 in the first worn state. The second controller 76 is, for example, configured to control the display 92 so as not to start displaying the electric power level in a case where the head protection device 70 is changed from the rest state to a state differing from the rest state in the first worn state.

The controller 76 is, for example, configured to control the display 92 to start displaying the electric power level in a case where the worn state is changed from the first worn state to the second worn state. The controller 76 is, for example, configured to control the display 92 to finish displaying the electric power level in a case where the worn state is changed from the first worn state to the second worn state and then the head protection device 70 enters the rest state.

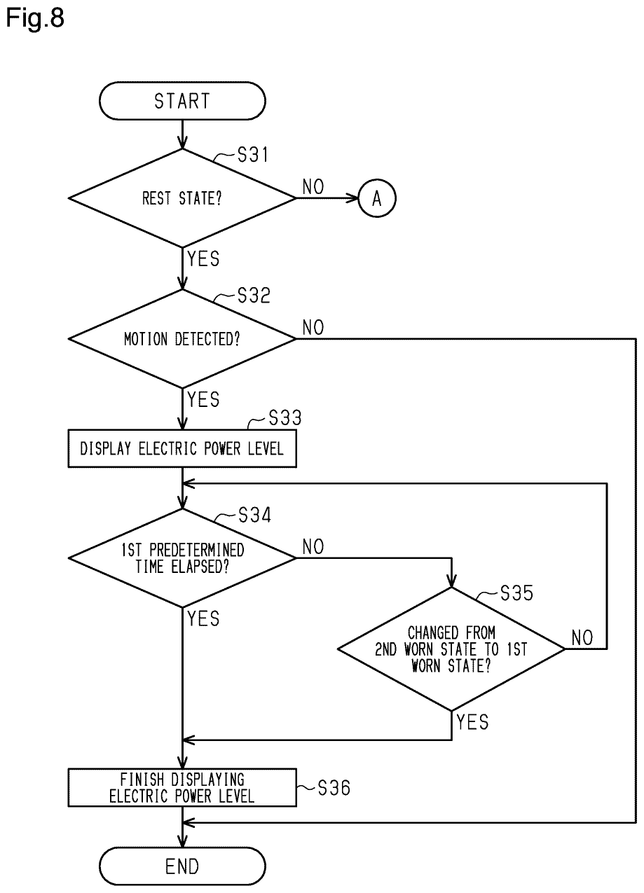

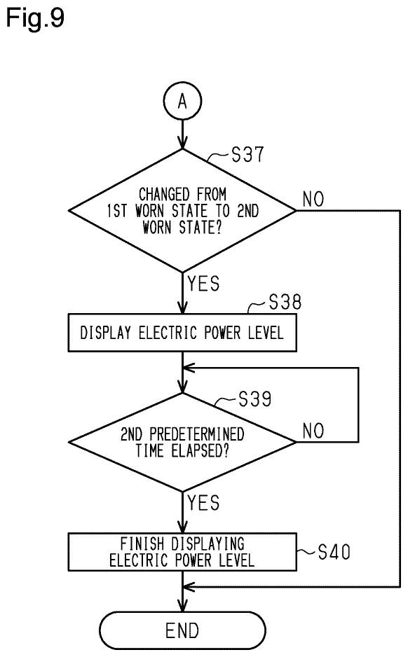

With reference to FIGS. 8 and 9, a control process for controlling the display 92 with the second controller 76 will be described. For example, in a case where electric power is supplied to the second controller 76, the second controller 76 starts the control process of the flowchart shown in FIG. 8 from step S31. In a case where the flowcharts shown in FIGS. 8 and 9 end, the second controller 76 repeats the control process from step S31 after a predetermined interval, for example, until the supply of electric power stops.

In step S31, the second controller 76 determines whether the head protection device 70 is in the rest state. In an example, in a case where the worn state is the first worn state, the second controller 76 determines that the head protection device 70 is not in the rest state. The second controller 76 can be configured to determine that the head protection device 70 is in the rest state in a case where the worn state is the second worn state and the electric power level is not displayed on the display 92. The second controller 76 can be configured to determine the head protection device 70 is in the rest state in a case where a predetermine time elapses from when the worn state is changed from the first worn state to the second worn state. In a case where the head protection device 70 is in the rest state, the second controller 76 proceeds to step S32.

In step S32, the second controller 76 determines whether motion is detected. For example, in a case where a detection value of the motion detector 94 is greater than or equal to a first predetermined value and a case where the detection value of the motion detector 94 continues to be greater than or equal to a second predetermined value for a first predetermined time or longer, the second controller 76 determines that motion is detected. In a case where motion is not detected, the second controller 76 ends the control process. In a case where motion is detected, the second controller 76 proceeds to step S33.

In step S33, the second controller 76 displays the electric power level on the display 92 and then proceeds to step S34. In step S34, the second controller 76 determines whether the first predetermined time has elapsed. In step S33, for example, in a case where the first predetermined time has elapsed from when the electric power level is displayed on the display 92, the second controller 76 determines that the first predetermined time has elapsed. In a case where the first predetermined time has elapsed, the second controller 76 proceeds to step S36. In a case where the first predetermined time has not elapsed, the second controller 76 proceeds to step S35. In step S35, the second controller 76 determines whether the worn state is changed from the second worn state to the first worn state. In a case where the worn state is not changed from the second worn state to the first worn state, the second controller 76 proceeds to step S34. In a case where the worn state is changed from the second worn state to the first worn state, the second controller 76 proceeds to step S36.

In step S36, the second controller 76 controls the display 92 to finish displaying the electric power level and then ends the control process. Steps S34 and S35 allow the second controller 76 to finish displaying the electric power level in at least one of a case where it is determined that the first predetermined time has elapsed and a case where it is determined that the worn state is changed from the second worn state to the first worn state.

In step S31, in a case where the state differs from the rest state, the second controller 76 proceeds to step S37. In step S37, the second controller 76 determines whether the worn state is changed from the first worn state to the second worn state. In a case where the worn state is not changed from the first worn state to the second worn state, the second controller 76 ends the control process. In a case where the worn state is changed from the first worn state to the second worn state, the second controller 76 proceeds to step S38.

In step S38, the second controller 76 displays the electric power level on the display 92 and then proceeds to step S39. In step S39, the second controller 76 determines whether the second predetermined time has elapsed. In a case where the second predetermined time has not elapsed, the second controller 76 repeats step S39. In a case where the second predetermined time has elapsed, the second controller 76 proceeds to step S40. In step S40, the second controller 76 controls the display 92 to finish displaying the electric power level and then ends the control process. In the present embodiment, the second predetermined time differs from the first predetermined time. However, the second predetermined time can be the same as the first predetermined time.

### Third Embodiment

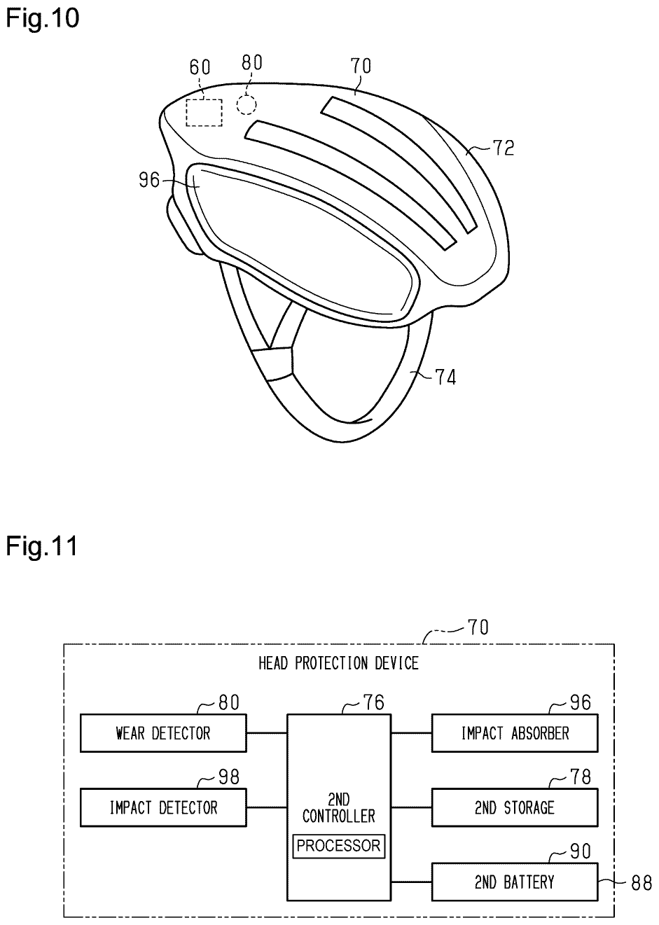

With reference to FIGS. 10 to 13, a third embodiment of the human-powered vehicle head protection device 70 will be described. In the third embodiment of the head protection device 70, the same reference characters are given to those elements that are the same as the corresponding elements of the first and second embodiments. Such elements will not be described in detail.

The head protection device 70 of the present embodiment includes a wear detector 80 configured to detect the worn state of the head protection device 70 on the rider, an impact absorber 96, and a controller 76 configured to control the impact absorber 96. In the present embodiment, the second communication unit 86 can be omitted from the head protection device 70.

The head protection device 70 further includes, for example, an impact detector 98. In an example, in a case where the impact detector 98 detects an impact, the second controller 76 actuates the impact absorber 96 to absorb the impact on the rider. The impact absorber 96 includes, for example, an air bag. The air bag is, for example, provided on at least one of an outer peripheral surface and a top surface of the main body 72. The air bag can be provided on a surface of the main body 72 facing the body of the rider.

The controller 76 is, for example, configured to control the impact absorber 96 based on the worn state detected by the wear detector 80. The controller 76 is, for example, configured to control the impact absorber 96 in either one of a first actuation state in which the impact absorber 96 is actuated and a second actuation state in which the impact absorber 96 is actuated in a manner more limited than in the first actuation state. The controller 76 is, for example, configured to control the impact absorber 96 in the second actuation state in a case where the head protection device 70 is not worn. In a case where the impact absorber 96 is in the second actuation state, the second controller 76 is, for example, configured to control the impact absorber 96 so as not to actuate the impact absorber 96 even in a case where the impact detector 98 detects an impact. In a case where the worn state is the second worn state, the second controller 76 can be configured to control the impact detector 98 to stop the impact detector 98 from detecting an impact, thereby avoiding actuation of the impact absorber 96.

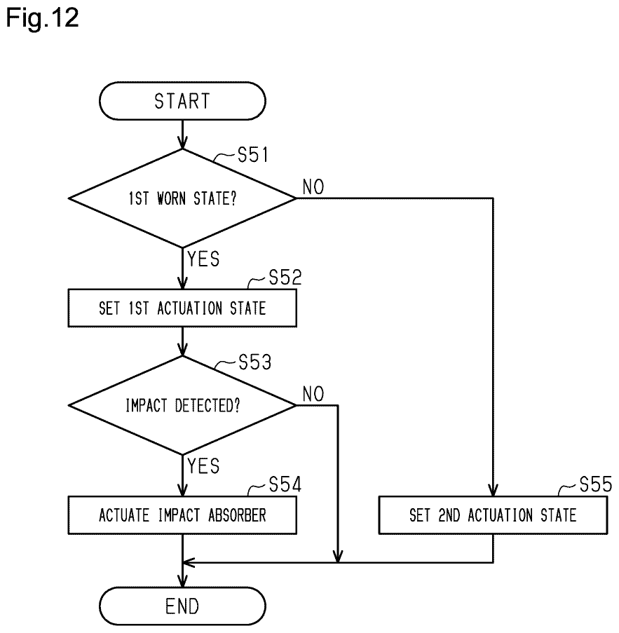

With reference to FIG. 12, a control process for controlling the impact absorber 96 with the second controller 76 will be described. For example, in a case where electric power is supplied to the second controller 76, the second controller 76 starts the FIG. 12 process of the flowchart shown in FIG. 12 from step S51. In a case where the flowchart shown in FIG. 12 ends, for example, the second controller 76 repeats the control process from step S51 after a predetermined interval until the supply of electric power stops.

In step S51, the second controller 76 determines whether the worn state is the first worn state. In a case where the worn state is the first worn state, the second controller 76 proceeds to step S52. In step S52, the second controller 76 sets the actuation state of the impact absorber 96 to the first actuation state and then proceeds to step S53.

In step S53, the second controller 76 determines whether an impact is detected. In a case where an impact is not detected, the second controller 76 ends the control process. In a case where an impact is detected, the second controller 76 proceeds to step S54. In step S54, the second controller 76 actuates the impact absorber 96 and then ends the control process.

In step S51, in a case where the worn state is not the first worn state, the second controller 76 proceeds to step S55. In step S55, the second controller 76 sets the actuation state of the impact absorber 96 to the second actuation state.

The second controller 76 can be configured to determine whether the worn state is the first worn state or the second worn state based on a worn state stored in the second storage 78. The second controller 76 can be, for example, configured to control the impact absorber 96 based on a determination result of the worn state based on the worn state stored in the second storage 78.

The second controller 76 is, for example, configured to store, in the second storage 78, a determination result of the worn state in accordance with a reception count of a wearing signal received from the wear detector 80. In an example, in a case where the rider is wearing the head protection device 70, the wear detector 80 can be configured to transmit a wearing signal to the second controller 76 in each predetermined cycle. The predetermined cycle is equal to, for example, the detection cycle of the wear detector 80. The second controller 76 can be configured to determine the worn state in accordance with the reception count of the wearing signal from the wear detector 80. The second controller 76 can be configured to control the impact absorber 96 based on a determination result of the worn state in accordance with the reception count of the wearing signals from the wear detector 80.

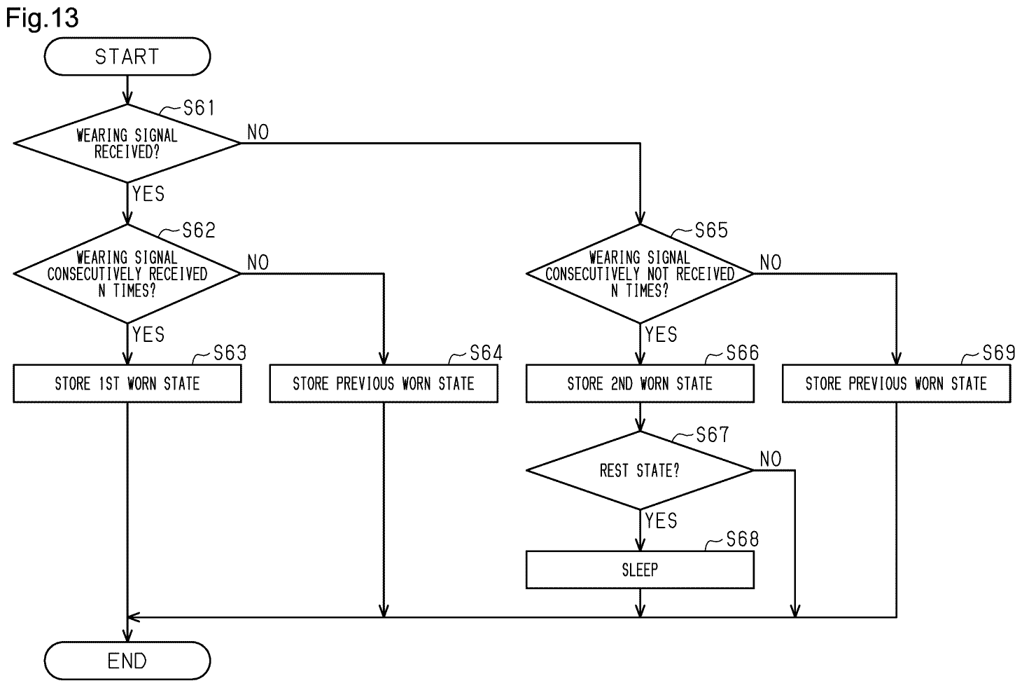

With reference to FIG. 13, a control process for determining the worn state in accordance with the reception count of the wearing signal from the wear detector 80 with the second controller 76 will be described. For example, in a case where electric power is supplied to the second controller 76, the second controller 76 starts the control process of the flowchart shown in FIG. 13 from step S61. In a case where the flowchart shown in FIG. 13 ends, for example, the second controller 76 repeats the control process from step S61 after a predetermined interval until the supply of electric power stops.

In step S61, the second controller 76 determines whether the wearing signal is received from the wear detector 80. In a case where the wearing signal is received from the wear detector 80, the second controller 76 proceeds to step S62. In step S62, the second controller 76 determines whether the wearing signal is consecutively received N times. In this case, N is any natural number. In a case where the wearing signal is consecutively received N times, the second controller 76 proceeds to step S63. In step S63, the second controller 76 stores the first worn state and ends the control process. The second controller 76 stores, for example, information indicating that the current worn state is the first worn state in the second storage 78.

In step S62, in a case where the wearing signal is not consecutively received N times, the second controller 76 proceeds to step S64. In step S64, the second controller 76 stores the previous worn state and then ends the control process. The second controller 76 stores, for example, information indicating that the current worn state remains the same as that saved at the previous time in the second storage 78.

In step S61, in a case where the wearing signal is not received from the wear detector 80, the second controller 76 proceeds to step S65. In step S65, the second controller 76 determines whether the wearing signal is consecutively not received N times. In an example, in a case where the wearing signal is not received since the previous Nth reception cycle of the wearing signal, the second controller 76 determines YES in step S65. In an example, in a case where the wearing signal is received at least once since the previous Nth reception cycle of the wearing signal, the second controller 76 determines NO in step S65. In the case of YES in step S65, the second controller 76 proceeds to step S66.

In step S66, the second controller 76 saves the second worn state and proceeds to step S67. The second controller 76 stores, for example, information indicating that the current worn state is the second worn state in the second storage 78. In step S67, the second controller 76 determines whether the head protection device 70 is in the rest state. In a case where the head protection device 70 is in the rest state, the second controller 76 proceeds to step S68. In step S68, the second controller 76 sets the second controller 76 to a sleep state and then ends the control process. In step S67, in a case where the head protection device 70 is not in the rest state, the second controller 76 ends the control process without proceeding to step S68.

In the case of NO in step S65, the second controller 76 proceeds to step S69. The second controller 76 stores the previous worn state in step S69 and then ends the control process. The second controller 76 stores, for example, information indicating that the current worn state remains the same as that saved at the previous time in the second storage 78.

### Fourth Embodiment

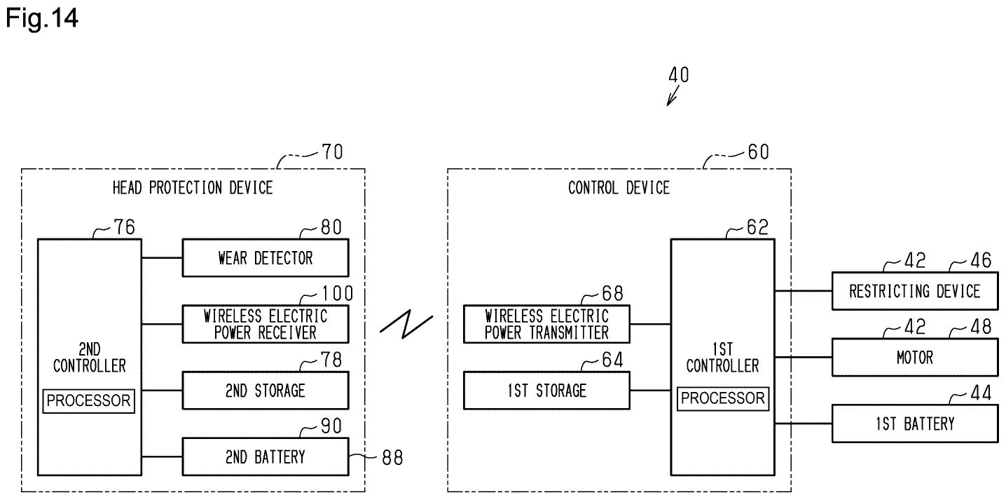

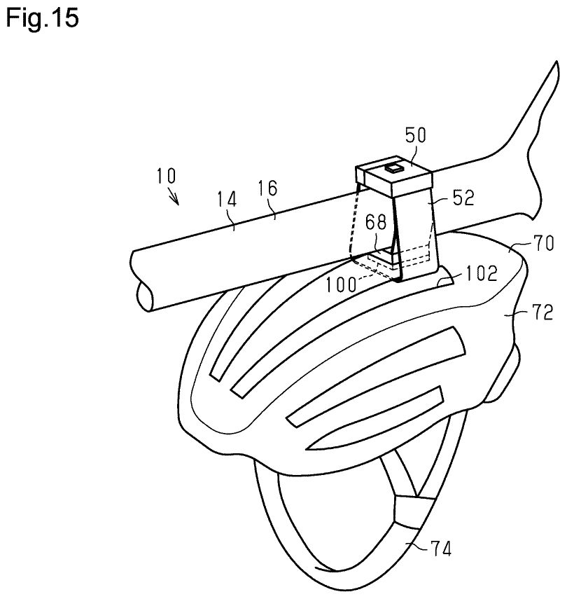

With reference to FIGS. 14 and 15, a fourth embodiment of the human-powered vehicle head protection device 70 will be described. In the fourth embodiment of the head protection device 70, the same reference characters are given to those elements that are the same as the corresponding elements of the first, second, and third embodiments. Such elements will not be described in detail.

In the present embodiment, the head protection device 70 further includes a wireless electric power receiver 100. The wireless electric power receiver 100 is configured to receive electric power through wireless power supply. The electric power received by the wireless electric power receiver 100 is charged to, for example, the second battery 90. The wireless electric power receiver 100 is, for example, provided on the main body 72.

The control device 60 further includes, for example, a wireless electric power transmitter 68. The wireless electric power transmitter 68, for example, transmits electric power to the wireless electric power receiver 100. The first controller 62 is, for example, configured to control the wireless electric power transmitter 68 to transmit electric power to the wireless electric power receiver 100. The wireless electric power transmitter 68 is, for example, provided on the frame 16. The wireless electric power transmitter 68 can be provided on the first battery 44.

The control system 40 can include, for example, a holder 50 that holds the head protection device 70 on the human-powered vehicle 10. The holder 50 is, for example, configured to hold the head protection device 70 close to the wireless electric power transmitter 68. The wireless electric power transmitter 68 can be provided on the holder 50. In a case where the wireless electric power transmitter 68 is provided on the holder 50, for example, the holder 50 is mounted on the frame 16 so that the wireless electric power transmitter 68 is provided on the frame 16.

The head protection device 70 further includes, for example, a position adjuster 102 configured to adjust a position of the wireless electric power receiver 100 relative to the wireless electric power transmitter 68 that supplies electric power to the wireless electric power receiver 100. The position adjuster 102 is, for example, provided on the main body 72. The position adjuster 102 is, for example, configured to be attached to the holder 50. The holder 50, for example, includes an attachment member 52 for attaching the head protection device 70 to the human-powered vehicle 10. The attachment member 52 is, for example, configured to engage with the position adjuster 102. The attachment member 52 is, for example, belt-shaped. The position adjuster 102 includes, for example, a hole that allows for insertion of the attachment member 52. The position adjuster 102 can be, for example, formed integrally with vent holes in the main body 72. The attachment member 52 is inserted into the position adjuster 102 so that in a state where the wireless electric power receiver 100 is located at a position corresponding to the wireless electric power transmitter 68 and the head protection device 70 is attached to the human-powered vehicle 10.

### Modifications

The description related to the above embodiments exemplifies, without any intention to limit, applicable forms of a human-powered vehicle control device, a human-powered vehicle control system, and a head protection device head protection device. The human-powered vehicle control device, the human-powered vehicle control system, and the human-powered vehicle head protection device according to the present disclosure can be applied to, for example, modifications of the embodiments that are described below and combinations of at least two of the modifications that do not contradict each other. In the modifications described hereafter, same reference characters are given to those elements that are the same as the corresponding elements of the above embodiments. Such elements will not be described in detail.

At least a portion of the first controller 62 can be provided on the head protection device 70. In a case where the first controller 62 is provided on the head protection device 70, at least a portion of the first controller 62 can be formed integrally with the second controller 76. In the modification, the first controller 62 can be, for example, configured to perform wireless communication with the component 42.

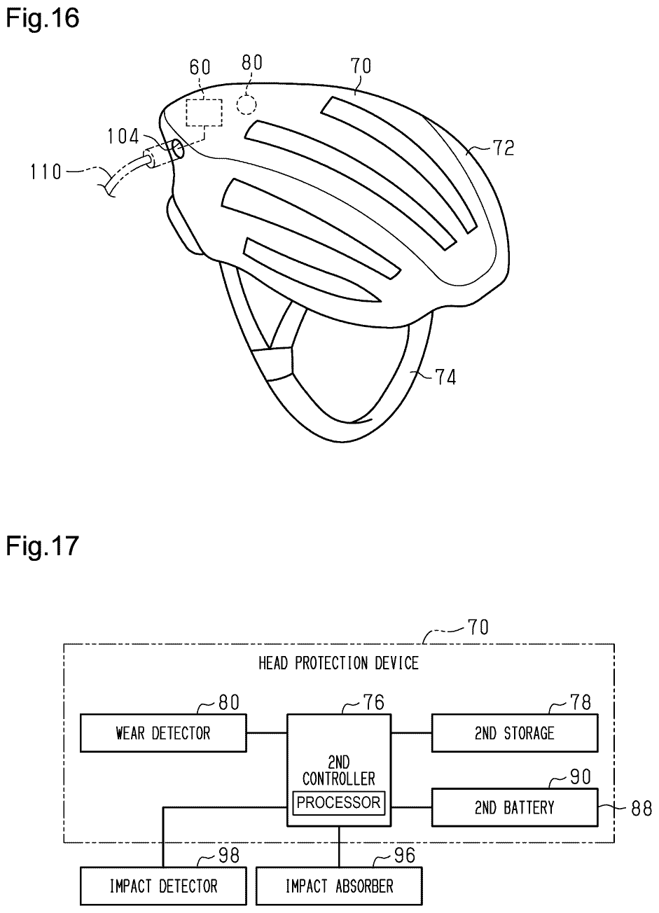

As shown in FIG. 16, the head protection device 70 can further include an electric power wire receiver 104 to which a power supply line 110 is attached. In the modification shown in FIG. 16, the wireless electric power receiver 100 can be omitted from the head protection device 70.

In the third embodiment, as shown in FIG. 17, at least one of the impact absorber 96 and the impact detector 98 can be formed separately from the head protection device 70. In this modification, for example, the head protection device 70 is configured to be worn on the head. In addition, for example, the impact absorber 96 is configured to be worn on a part of the rider other than the head.

In the first and second embodiments, the second controller 76 can be configured to determine whether the worn state is the first worn state or the second worn state based on a worn state stored in the second storage 78. The second controller 76 can be, for example, configured to control the impact absorber 96 based on a determination result of the worn state based on the worn state stored in the second storage 78. In this modification, for example, the worn state can be determined through the control process of the flowchart of the third embodiment shown in FIG. 13.

The component 42 can include, for example, at least one of a transmission device, a braking device, an adjustable seatpost, a suspension, a lamp, a display device, and the battery 44 instead of or in addition to at least one of the restricting device 46 and the motor 48. The display device includes, for example, a cycle computer. In a case where the component 42 includes the display device, the first controller 62 can be configured to control the display device to display the electric power level of the second battery 90 in a case where the worn state is the first worn state.

In this specification, the phrase “at least one of” as used in this disclosure means “one or more” of a desired choice. For one example, the phrase “at least one of” as used in this disclosure means “only one single choice” or “both of two choices” if the number of its choices is two. For another example, the phrase “at least one of” as used in this disclosure means “only one single choice” or “any combination of equal to or more than two choices” if the number of its choices is equal to or more than three. Also, the term “and/or” as used in this disclosure means “either one or both of”. For instance, the phrase “at least one of A and B” encompasses (1) A alone, (2), B alone, and (3) both A and B. The phrase “at least one of A, B, and C” encompasses (1) A alone, (2), B alone, (3) C alone, (4) both A and B, (5) both B and C, (6) both A and C, and (7) all A, B, and C. In other words, the phrase “at least one of A and B” does not mean “at least one of A and at least one of B” in this disclosure.

In this specification, ordinal numbers such as “first, second, and third” are used to merely distinguish between members having the same name and have no particular meaning.

What is claimed is:

1. A control device for a human-powered vehicle, the control device comprising:
a controller configured to control a component of the human-powered vehicle based on a worn state of a head protection device that is wearable by a rider of the human-powered vehicle.

2. The control device according to claim 1, further comprising:
a receiver configured to receive predetermined information related to the worn state from the head protection device,
the controller being configured to control the component based on the predetermined information.

3. The control device according to claim 1, wherein
the component includes a restricting device configured to restrict traveling of the human-powered vehicle, and
the controller is configured to control the restricting device based on the worn state.

4. The control device according to claim 1, wherein
the component includes a motor configured to apply a propulsion force to the human-powered vehicle, and
the controller is configured to control the motor based on the worn state.

5. The control device according to claim 1, wherein
the controller is configured to change an electric power consumption state of the component based on the worn state.

6. A control system for the human-powered vehicle, the control system comprising:
the control device according to claim 1; and
a human-powered vehicle head protection device including a transmitter configured to transmit predetermined information related to the worn state to the control device.

7. A human-powered vehicle head protection device that is wearable by a rider of a human-powered vehicle, the head protection device comprising:
a wear detector configured to detect a worn state of the head protection device that is worn by the rider; and
a transmitter configured to transmit predetermined information related to the worn state, detected by the wear detector, to an external device.

8. A human-powered vehicle head protection device that is wearable by a rider of a human-powered vehicle, the head protection device comprising:
a power supply;
a wear detector configured to detect a worn state of the head protection device that is worn by the rider;
a display configured to display an electric power level of the power supply; and
a controller configured to control the display,
the controller being configured to control the display to change a display state of the electric power level on the display based on the worn state detected by the wear detector.

9. The head protection device according to claim 8, wherein
the worn state includes a first worn state in which the head protection device is worn by the rider and a second worn state in which the head protection device is not worn by the rider, and
the controller is configured to control the display to change the display state in a case where the worn state is changed from one of the first worn state and the second worn state to the other one of the first worn state and the second worn state.

10. The head protection device according to claim 9, further comprising:
a motion detector configured to detect motion of the head protection device,
the controller being configured to control the display to start displaying the electric power level in a case where motion of the head protection device is detected by the motion detector in the second worn state.

11. The head protection device according to claim 10, wherein
the motion detector includes at least one of an acceleration sensor and a gyro sensor.

12. The head protection device according to claim 11, wherein
in a case where the worn state changes from the second worn state to the first worn state, the controller is configured to control the display to finish displaying the electric power level.

13. The head protection device according to claim 9, wherein
in a case where the worn state changes from the first worn state to the second worn state, the controller is configured to control the display to start displaying the electric power level.

14. A human-powered vehicle head protection device that is wearable by a rider of a human-powered vehicle, the head protection device comprising:
a wear detector configured to detect a worn state of the head protection device that is worn by the rider;
an impact absorber; and
a controller configured to control the impact absorber,
the controller being configured to control the impact absorber based on the worn state detected by the wear detector.

15. The head protection device according to claim 14, wherein
the controller is configured to control the impact absorber in either one of a first actuation state in which the impact absorber is actuated and a second actuation state in which the impact absorber is actuated in a manner more limited than in the first actuation state, and
the controller is configured to control the impact absorber in the second actuation state in a case where the head protection device is not worn.

16. The head protection device according to claim 7, further comprising
a wireless power receiver.

17. The head protection device according to claim 16, further comprising
a position adjuster configured to adjust a position of the wireless power receiver relative to a wireless power transmitter that supplies electric power to the wireless power receiver.

18. The head protection device according to claim 7, further comprising
a wired power receiver to which an electric power wire is connected.

19. The head protection device according to claim 7, wherein
the wear detector is configured to detect a position of a head of the rider relative to the head protection device.

20. The head protection device according to claim 19, wherein
the wear detector is configured to detect the position based on information from a sensor that detects a reflected signal.

21. The head protection device according to claim 7, further comprising:
a belt configured to attach the head protection device to the rider,
the wear detector being configured to detect a coupled state of the belt.
